(19) United States
(12) Patent Application Publication
(10) Pub. No.: US 2024/0153082 A1
(43) Pub. Date: May 9, 2024

(54) DEEP LEARNING MODEL FOR DIAGNOSIS OF HEPATOCELLULAR CARCINOMA ON NON-CONTRAST COMPUTED TOMOGRAPHY

(71) Applicants: Versitech Limited, Hong Kong (CN); The Education University of Hong Kong, Hong Kong (CN)

(72) Inventors: Chengzhi Peng, Hong Kong (CN); Leung Ho Philip Yu, Hong Kong (CN); Wan Hang Keith Chiu, Hong Kong (CN); Xianhua Mao, Hong Kong (CN); Man Fung Yuen, Hong Kong (CN); Wai Kay Walter Seto, Hong Kong (CN)

(21) Appl. No.: 18/471,971

(22) Filed: Sep. 21, 2023

Related U.S. Application Data
(60) Provisional application No. 63/382,198, filed on Nov. 3, 2022.

(51) Int. Cl. G06T 7/00; A61B 6/00; A61B 6/03; G06V 10/764; G06V 10/82; G06V 20/50; G16H 30/40; G16H 50/20

(52) U.S. Cl. CPC G06T 7/0012; A61B 6/032; A61B 6/50; G06V 10/764; G06V 10/82; G06V 20/50; G16H 30/40; G16H 50/20; G06T 2200/24; G06T 2207/10081; G06T 2207/20081; G06T 2207/20084; G06T 2207/30056; G06T 2207/30096; G06V 2201/03

(57) ABSTRACT

Disclosed is a computer-implemented three-dimensional image classification system (CIS) for processing and/or analyzing non-contrast computed tomography (CT) medical imaging data. The CIS is a deep neural network containing multiple Convolutional Block Attention Module (CBAM) blocks, which contain convolutional layers for feature extraction followed by CBAMs. The CBAM applies channel attention to highlight more relevant features and spatial attention to focus on more important regions. Max pooling layers operably link adjacent pairs of CBAM blocks. The output of the final CBAM block is passed to two terminal fully connected layers to generate a diagnosis. This classification system can be used to perform efficient diagnosis of hepatocellular carcinoma using solely non-contrast CT images, with diagnostic performance comparable to that of a radiologist using the current LIRADS system.

### CROSS-REFERENCE TO RELATED APPLICATIONS

This application claims benefit of U.S. Provisional Application No. 63/382,198 “DEEP LEARNING MODEL FOR DIAGNOSIS OF HEPATOCELLULAR CARCINOMA ON NON-CONTRAST COMPUTED TOMOGRAPHY”, filed Nov. 3, 2022, by Chengzhi Peng, Leung Ho Philip Yu, Wan Hang Keith Chiu, Xianhua Mao, Man Fung Yuen, and Wai Kay Walter Seto, and is hereby incorporated herein by reference in its entirety.

### FIELD OF THE INVENTION

This invention is generally related to processing and visualizing data, particularly a computer-implemented system/method for processing and visualizing images of liver tissue in clinical settings, such as non-contrast computed tomography images to determine the presence of hepatocellular carcinoma.

### BACKGROUND OF THE INVENTION

Liver cancer poses a heavy burden of public health around the world. Among all cancers, it ranks sixth in incidence and third in mortality, with over 90,000 incident cases and more than 83,000 deaths annually worldwide (Global Cancer Observatory, 2021). Over the past decades, the overall age-standardized incidence rate of liver cancer has steadily grown, with an average of 0.34% per year from 1990 to 2016 (Liu, et al., J Hepatol 2019, 70(4):674-683). Liver cancer remains the major health concern for the past decades.

Hepatocellular carcinoma (HCC) is the most common type of liver malignancy (Mittal & El-Serag, Journal of Clinical Gastroenterology 2013, 47(0):S2-S6). Most patients with HCC present insidious clinical courses, being asymptomatic in the early stage, only presenting later with nonspecific symptoms such as abdominal pain, weight loss, and anorexia (Cahill & Braccia, Clinical Journal of Nursing Oncology 2004, 8(4):393-399; Teo & Fock, Digestive Diseases 2001, 19(4):263-268). This usually leads to significant delays in accurate diagnosis, accompanied by more severe disease stages and untimely treatment. Furthermore, the overall survival of HCC depends greatly on the disease stage, from the 1-year survival rate ranges of 80% for stage IA to only 10% for stage IV disease (Lin, et al., PLoS ONE 2020, 15(10):e0240542). Therefore, early diagnosis is especially crucial for improving the treatment and prognosis of HCC.

In clinical practices, routine diagnosis of HCC is commonly conducted via computed tomography (CT) and magnetic resonance imaging (MRI), under a standardized diagnostic framework known as the Liver Imaging Reporting and Data System (LI-RADS) (Bargellini, et al., Journal of Hepatocellular Carcinoma 2014, 1:137-148). The LI-RADS system classifies liver lesions according to multiple radiological features, most importantly arterial phase hyperenhancement and nonperipheral washout in the portal-venous or delayed phase, yielding the LI-RADS 5 categories (LR-1 to LR-5) of HCC (American College of Radiology 2018). While the LI-RADS system demonstrates superior diagnostic performance in LR-5 (100% definite HCC), several limitations also exist. The most notable is that the intermediate categories (i.e., LR-2, LR-3 and LR-4) do not provide a definitive diagnosis, with the recommended management being frequent surveillance or repeat diagnostic imaging, which can lead to delays in diagnosis and treatment (Kim, et al., European Radiology 2018, 29:1124-1132).

According to the latest guidelines by the American Association for the Study of Liver Diseases (AASLD) and the European Association for the Study of the Liver (EASL), multiphase CT scans have a pivotal role in the diagnosis of HCC during routine clinical practices (Marrero, et al., Hepatology 2018, 68(2):723-750; European Association for the Study of the Liver, Hepatology 2018, 69(1):182-236). The multiple phases of CT slices are usually taken at different time points in terms of iodinated contrast media injection: the non-contrast phase (taken before contrast injection); the arterial phase (taken at 15-35 seconds after injection), the portal-venous phase (60-90 seconds after injection), and the delayed phase (around 10 minutes after injection). In general, contrast injection is necessary to enhance the intensity difference between normal tissues and tumors since many tumors have attenuation levels similar to that of the normal liver. However, radiological contrast may cause a wide range of adverse reactions, from mild flushing, nausea, and vomiting to contrast-induced nephropathy (CIN) and even life-threatening anaphylactic events (Hasebroock & Serkova, Expert Opinion and Drug Metabolism and Toxicology 2009, 5(4):403-416). In addition, contrast agents may interact with commonly used pharmacological agents, limiting their application in specific patients. Therefore, the development of a diagnostic system based on non-contrast CT would avoid adverse effects of contrast agents, reduce the level of radiation exposure, shorten the duration of the CT scan, and potentially allow non-contrast CT to be employed as a screening tool for HCC.

Traditionally, radiological images were examined by radiologists, who manually go over the slices to detect individual lesions and arrive at a diagnosis. This process relies greatly on the experience and education of the radiologist and often can be subjective (Hosny, et al., Nature Reviews Cancer 2018, 18:500-510). These limitations call for a new and effective assisted strategy or system. In recent years, the application of artificial intelligence (AI) has become increasingly popular in medicine, especially in the field of medical imaging. Many investigations, applying algorithms of AI, machine learning, and deep learning, have been explored to enable automated diagnosis of HCC via CT images. Li, et al. (Li, et al., Biocybernetics and Biomedical Engineering 2020, 40(1):238-248) proposed a computer-aided diagnostic system (CAD), including a fully convolutional network (FCN) based on a fine-tuned VGG-16 model for liver and tumor segmentation, as well as a 9-layer convolutional neural network (CNN) for HCC classification. Shi, et al. (Shi, et al., Abdominal Radiology 2020, 45:2688-2697) used a 2.5-dimensional, multiphase convolutional network (MP-CDN) for binary classification of focal liver lesions into HCC versus non-HCC. Wang, et al. (Wang, et al., British Journal of Cancer 2021, 125(8): 1111-1121) developed two 34-layer residual convolutional networks named NoduleNet and HCCNet for classification of HCC versus non-HCC. Vivanti, et al. (Vivanti, et al., International Journal of Computer Assisted Radiology and Surgery 2017, 12:1945-1957) proposed a CNN classifier for detection and segmentation of new tumors in follow-up CT scans, employing other machine learning methods such as Random Forest and Support Vector Machine for classification. Chlebus, et al. (Chlebus, et al., Scientific Reports 2018, 8:15497) used a U-Net like fully convolutional neural network for tumor segmentation and detection, additionally employing several object-based preprocessing steps to enhance tumor detection performance. Liang, et al. (Liang, et al., Combining Convolutional and Recurrent Neural Networks for Classification of Focal Liver Lesions in Multi-phase CT Images. In: Frangi A, Schnabel J, Davatzikos C, Alberola-López C, Fichtinger G. (eds) Medical Image Computing and Computer Assisted Intervention—MICCAI 2018. Lecture Notes in Computer Science 2018, 11071:666-675) combined a residual network (ResNet) with global and local pathways (ResGLNet) and a bidirectional long short-term memory (BD-LSTM) model, developing a ResGL-BD-LSTM model for the task of liver lesion classification; they also developed a new loss function composed of an inter-loss and intra-loss for model training. Gao, et al. (Gao, et al., Journal of Hematology and Oncology 2021, 14:154) developed a SpatialExtractor-TemporalEncoder-Integration-Classifier (STIC) model for classification of malignant hepatic tumors, using a VGG16 network to extract and encode features and then passing on the output to a temporal encoder combined with clinical data. As shown by the large volume of studies and their recency, there exists a large unmet need for the development of accurate and efficient methods for diagnosis of medical conditions such as liver cancers particularly HCC, using medical images particularly CT scans.

While the aforementioned methods have achieved satisfactory diagnostic performance, they mostly rely on multi-phase, contrast-enhanced CT scans, which have many limitations as previously described. Regarding non-contrast CT scans, the number of studies investigating the potential of using solely non-contrast CT scans for HCC diagnosis is limited. Yasaka, et al. (Yasaka, et al., Radiology 2018, 286(3):887-896) used a simple CNN with 6 convolutional layers for the classification of liver lesions into five categories. This involved five models using different combinations of CT phases, including a model using only unenhanced or non-contrast scans. However, the unenhanced scans gave a suboptimal diagnostic performance with a test accuracy of 0.48 and an area under curve (AUC) of 0.61. Cheng, et al. (Cheng, et al., Hepatology Communications 2022, 6(10):2901-2913) developed a flexible three-dimensional deep learning algorithm, heterophase volumetric detection (HPVD), an extension of another algorithm known as volumetric universal lesion detection (VULD). These algorithms generate bounding boxes in multiphase 3D CT inputs, and then further makes a patient-level classification. When evaluated on a dataset of 164 positives and 206 controls, the VULD model and HPVD models achieved AUCs of 0.66 and 0.71, respectively. Evidently, more innovative and effective models are needed to achieve the task of accurate HCC diagnosis using non-contrast CT scans, which will have significant implications on the clinical application of CT in many situations.

Therefore, it is an object of this invention to build and provide a deep neural network-based algorithm to achieve improved accurate diagnostic performance of a disease using only non-contrast CT images of tissues or organs of interest.

It is also an object of this invention to build and provide a deep neural network-based algorithm to achieve improved accurate diagnostic performance of HCC using only non-contrast CT images of the liver.

### SUMMARY OF THE INVENTION

Described herein is a computer-implemented systems (CIS) or a Convolutional Block Attention Module (CBAM) model and computer-implemented methods (CIM) that use the CIS. The CIS or CBAM model contains a three-dimensional deep convolutional neural network for processing and classification of cross-sectional images. The CBAM model improves on traditional convolutional neural networks by incorporating attention mechanisms, allowing the model to focus on more differentiated and relevant features and spatial regions for more accurate performance. Here, and in the ensuring text, CIS and CBAM model are used interchangeably.

The CBAM model is composed of four CBAM blocks arranged in a series configuration. Each CBAM block contains one or more CBAMs, each of which contains a channel attention module and a spatial attention module.

In some forms, the classification model contains:
(i) A first CBAM block, a second CBAM block, a third CBAM block and a fourth CBAM block in a series configuration. Each CBAM block contains one or more CBAMs. Each CBAM contains a channel attention module and spatial attention module, arranged in a series configuration. The channel attention module contains two parallel pathways which first pass through a global average pooling layer and a global max pooling layer respectively, and then pass through two shared fully connected layers to extract the channel attention map. The channel attention map is multiplied with the original input. The spatial attention module contains an average pooling layer and a max pooling layer along the channel axis, followed by a concatenation layer and a 3D convolutional layer to extract the spatial attention map. The spatial attention map is multiplied with the original input;
(ii) Transition layers between the CBAM blocks, operably linked to pairs of CBAM blocks in the series configuration. The transition layer contains a max pooling layer with a stride size of (2, 2, 1) or (3, 3, 3); and
(iii) A classification layer operably linked to the fourth CBAM block. The classification layer contains a flattening layer and two terminal fully connected layers. The two fully connected layers contain a rectified linear unit (ReLu) activation function and a sigmoid activation function, respectively.

The CIS and CIM are not limited to any particular hardware or operating system and are useful for processing and/or analyzing medical imaging input data. In a preferred embodiment, the imaging input data are non-contrast CT liver scans. The CIS and CIM allow a user to make diagnoses or prognoses of a disease and/or disorder, based on output preferably displayed on a graphical user interface. A preferred disease and/or disorder includes HCC.

### BRIEF DESCRIPTION OF THE DRAWINGS

FIGS. 1A, 1B, and 1C are schematics of a complete CBAM classification model (FIG. 1A), a CBAM block (FIG. 1B), and a CBAM (FIG. 1C).

### DETAILED DESCRIPTION OF THE INVENTION

### I. Definitions

The term “CBAM” refers to a component in the neural network that applies attention mechanisms to an input image or feature map. The CBAM is composed of a channel attention module and a spatial attention module, arranged in a series configuration.

The term “CBAM block” refers to an integrated component of the neural network containing one or more CBAMs operably linked to convolutional layers, activation layers and batch normalization layers.

The term “CBAM model” refers to a complete end-to-end deep neural network for the classification of CT images. A CBAM model contains multiple CBAM blocks arranged in series, preferably linked by transitional max pooling layers, which receive input data from an input layer and pass on processed data to a fully connected network to compile the extracted features and obtain a prediction.

The term “activation function” describes a component of a neural network that may be used to bound neuron output, such as bounding between zero and one. Examples include a sigmoid activation function, Rectified Linear Unit (“ReLU”), or parametric rectified linear unit activation function (PReLu).

The term “convolutional layer” describes a component in a neural network that transforms data (such as input data) in order to retrieve features from it. In this transformation, the data (such as an image) is convolved using one or more kernels (or one or more filters).

The term “pooling layer” refers to a component in a neural network, that performs down-sampling for feature compression. The “pooling layer” can be a “max pooling” layer or an “average pooling” layer. “Down-sampling” refers to the process of reducing the dimensions of input data compared to its full resolution, while simultaneously preserving the necessary input information for classification purposes. Typically, coarse representations of the input data (such as image) are generated.

The term “kernel” refers to a surface representation that can be used to represent a desired separation between two or more groups. The kernel is a parameterized representation of a surface in space. It can have many forms, including polynomial, in which the polynomial coefficients are parameters. A kernel can be visualized as a matrix (2D or 3D), with its height and width smaller than the dimensions of the data (such as input image) to be convolved. The kernel slides across the data (such as input image), and a dot product of the kernel and the input data (such as input image) are computed at every spatial position. The length by which the kernel slides is known as the “stride length.” Where more than one feature is to be extracted from the data (such as input image), multiple kernels can be used. In such a case, the size of all the kernels are preferably the same. The convolved features of the data (such as input image) are stacked one after the other to create an output so that the number of channels (or feature maps) is equal to the number of kernels used.

### II. Computer-Implemented Systems and Methods

A computer-implemented (CIS) containing one or more Convoluted Block Attention Module (CBAM) blocks is described. The CIS contains a three-dimensional deep convolutional neural network for processing and classification of cross-sectional images. The complete end-to-end CIS, also known as a CBAM model, contains a classification deep learning-based algorithm for distinguishing between diseased and non-diseased tissues.

Apart from common convolutional layers used in traditional CNNs, attention mechanisms are applied in this CBAM model to enhance information processing. In brief, attention mechanisms allow the neural network to focus on the most critical and salient features, as well as spatial regions in a given input while filtering out irrelevant background information.

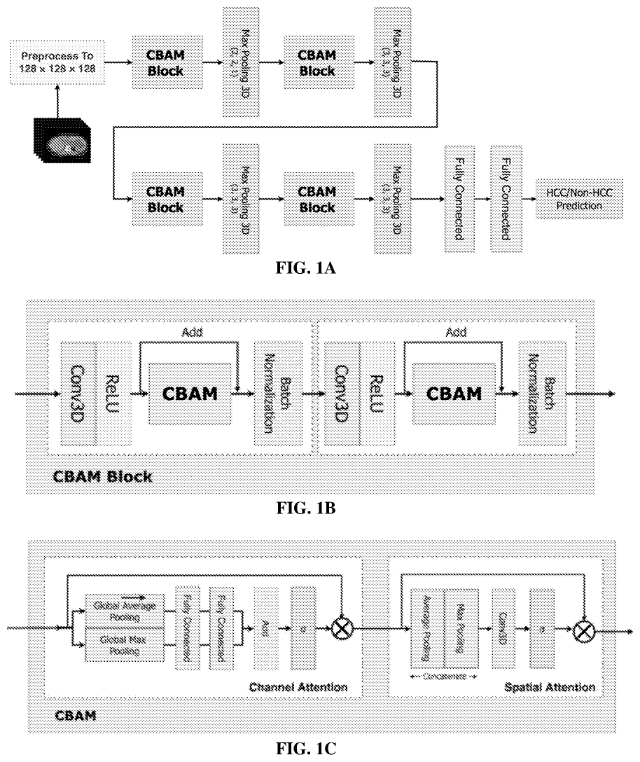

An overall, non-limiting architecture of a proposed CIS or CBAM model is shown in FIG. 1A. As shown, the CBAM model contains four CBAM blocks, arranged in a series configuration. Adjacent CBAM blocks are operably connected by transitional max pooling layers with pool sizes of (2, 2, 1) or (3, 3, 3), compressing the spatial dimensions of the feature maps. At the same time, the number of kernels in the convolutional layers progressively increases as one goes deeper in the neural network. Preferably, the number of filters contained in the convolutional layers in the first, second, third and fourth CBAM blocks are 128, 256, 512 and 1024 respectively. A large number of filters allows the CBAM model to capture a greater number of higher-level features, while also enhancing the effects of channel attention mechanisms, which can analyze and learn the dependencies between a larger number of channels. The three-dimensional output of the final CBAM block is passed through a flattening layer to become a one-dimensional vector, before being fed into a fully connected layer with a rectified linear unit (ReLU) activation function. This layer is connected to a final fully connected layer with one single unit, which outputs the probability of the CT scan containing diseased tissue, such as HCC lesions.

Further details on the CIS and components thereof are provided in the following sections.

i. Computer-Implemented System

A CIS or CBAM model that is not limited to any particular hardware or operating system is provided for processing and/or analyzing imaging and/or non-imaging input data is described. The CIS allows a user to make diagnoses or prognoses of a disease and/or disorder, based on output preferably displayed on a graphical user interface. A preferred disease and/or disorder includes HCC.

The CIS contains one or more CBAM blocks, and at least one of the one or more CBAM blocks contains a CBAM.

(a) CBAM

The CBAM contains a channel attention module and a spatial attention module. The channel attention module and the spatial attention module can be arranged in a parallel or in a series configuration. Preferably, the channel attention module and the spatial attention module are arranged in a series configuration. In some forms, the channel attention module is placed before the spatial attention module.

(1) Channel Attention Module

The channel attention module functions to compress the spatial dimension and allow the model to learn an appropriate weighting for the different channels within a feature map, assigning greater importance to more relevant features while paying less attention to irrelevant features.

A non-limiting architecture of a channel attention module is shown in FIG. 1C. In general, the channel attention module contains two parallel pathways which first pass preferably through a global average pooling layer and a global max pooling layer, respectively. Within the channel attention module, each of the two parallel pathways contains two fully connected layers. In some forms, the channel attention module contains an addition layer that combines the outputs from each of the two parallel pathways. In some forms, the channel attention module further contains a first activation function. The first activation function is selected from a sigmoid activation function, a rectified linear unit activation function (ReLu) layer, and/or a parametric rectified linear unit activation function (PReLu) layer, preferably a sigmoid activation function.

Within the channel attention module, input is transmitted through two parallel pathways, e.g., one of which contains a global average pooling layer and the other contains a global max pooling layer, and then further transmitted separately through two shared fully connected layers. Usually, the outputs of the two separate pathways are added together, transmitted through an activation function, such as a sigmoid activation function, and multiplied with the original input to the channel attention module.

(2) Spatial Attention Module

The spatial attention module serves to highlight the more informative regions in an input image or feature map, while suppressing less important regions.

A non-limiting architecture of a channel attention module is shown in FIG. 1C. In general, the spatial attention module contains a first pooling layer and a second pooling layer. Preferably, the first pooling layer and the second pooling layer are an average pooling layer and a max pooling layer, respectively. In some forms, the spatial attention module contains a concatenation layer and a 3D convolutional layer after the average pooling layer and the max pooling layer. In some forms, the spatial attention module further contains a second activation function, after the 3D convolutional layer. The second activation function is selected from a sigmoid activation function, a rectified linear unit activation function (ReLu) layer, and/or a parametric rectified linear unit activation function (PReLu) layer, preferably a sigmoid activation function.

Within the spatial attention module, the input feature map undergoes average pooling and max pooling along the channel axis in parallel, with the resulting feature maps concatenated. Preferably, this is followed by a convolutional layer with a filter, whose output is multiplied with the original input to the spatial attention module.

(b) CBAM Block

In some forms, the CIS contains one or more CBAM blocks, two or more CBAM blocks, three or more CBAM blocks, or four or more CBAM blocks. The CBAM blocks can be arranged in a series configuration, a parallel configuration, or a combination thereof. Preferably, the CBAM blocks are arranged in a series configuration.

A non-limiting architecture of a CBAM block is shown in FIG. 1B. In general, each CBAM block independently contains one or more CBAMs with features as described above. In some forms, each CBAM block independently contains two or more CBAMs with features as described above.

Preferably, within the CBAM block, a CBAM is preceded by a convolutional layer, preferably containing a third activation function. The third activation function is selected from a rectified linear unit activation function (ReLu) layer, a parametric rectified linear unit activation function (PReLu) layer, and/or a sigmoid activation function layer, preferably a rectified linear unit activation function (ReLu). Preferably, the CBAM is proceeded by a normalization layer. The normalization layer is selected from a batch normalization layer, a weight normalization layer, a layer normalization layer, an instance normalization layer, a group normalization layer, a batch renormalization layer, and/or a batch-instance normalization layer, preferably a batch normalization layer.

In some forms, CBAM blocks of the CIS contain convolutional layers. Preferably, subsequent CBAM blocks contain progressively more kernels than convolutional layers in prior CBAM blocks.

In some forms, the CIS contains a CBAM block and two fully connected layers, arranged in series configuration. Preferably, both the CBAM block and two fully connected layers are arranged together in series, FIG. 1A. In these forms, the CBAM block contains two CBAMs, each containing a channel attention module and a spatial attention module with features as described above. The channel attention module and the spatial attention modules are arranged in series configuration. Further, each CBAM is preceded by a convolutional layer containing a ReLU activation function, and followed by a batch normalization layer.

Preferably, adjacent CBAM blocks in the disclosed CIS are operably linked by a transitional layer. Preferably, the transitional layer contains a pooling layer. In some forms, the pooling layer contains a max pooling layer or an average pooling layer, preferably a max pooling layer. In some forms, the pooling layer has a stride size of (2, 2, 1) or (3, 3, 3).

In some forms, the CIS further contains a classification layer operably linked to a terminal CBAM block of the CIS. In some forms, the classification layer contains a flattening layer and the two fully connected layers. Preferably, the two fully connected layers are in a series configuration. Preferably, the two fully connected layers contain an activation function, such as a sigmoid activation function.

ii. Computer-Implemented Method

Also described is a computer-implemented method (CIM) for analyzing data, which involves using any of the CISs described above. Preferably, the CIM involves visualizing on a graphical user interface, output from these CISs. Visualizing this output facilitates the diagnosis, prognosis, or both, of a disease or disorder in a subject. The disease or disorder includes, but is not limited to, tumors (such as liver, brain, or breast cancer, etc), cysts, joint abnormalities, abdominal diseases, liver diseases, kidney disorders, neuronal disorders, or lung disorders. A preferred disease or disorder is hepatocellular carcinoma.

In some forms, the data are images from one or more biological samples. The input imaging data are preferably from medical imaging applications, including, but not limited to, non-contrast CT scans. Preferably, the images are internal body parts of a mammal. In some forms, the internal body parts are livers, brains, blood vessels, hearts, stomachs, prostates, testes, breasts, ovaries, kidneys, neurons, bones, or lungs. Preferred input imaging data are non-contrast CT liver scans.

### III. Methods of Using

The described CIS or CIM can be utilized to analyze data. The CIS or CIM is one of general applicability and is not limited to imaging data from a patient population in a specific geographical region of the world. Preferably, the data are imaging data, such as medical imaging data obtained using well-known medical imaging tools such as non-contrast CT scans. Preferably, the imaging data are non-contrast CT medical images. Within the context of medical imaging, the CIS or CIM can be employed in the diagnosis or prognosis of diseases or disorders. The disclosed CIS or CIM are particularly used in analyzing non-contrast CT medical images of intra-abdominal organs (such as intra-abdominal tumoral organs) or intra-abdominal tissues (such as intra-abdominal tumoral tissues), particularly HCC.

The disclosed CISs and CIMs can be further understood through the following enumerated paragraphs or embodiments.

1. A three-dimensional computer-implemented classification system (CIS) containing one or more Convoluted Block Attention Module (CBAM) blocks, wherein at least one of the one or more CBAM blocks contains a CBAM, wherein the CBAM contains a channel attention module and a spatial attention module, preferably wherein the channel attention module and the spatial attention module are arranged in a series configuration.

2. The CIS of paragraph 1, wherein the channel attention module is proceeded by the spatial attention module within the CBAM.

3. The CIS of paragraph 1 or 2, wherein the channel attention module contains two parallel pathways which first pass through a global average pooling layer and a global max pooling layer respectively.

4. The CIS of paragraph 3, wherein, within the channel attention module, each of the two parallel pathways contains two fully connected layers.

5. The CIS of paragraph 4, wherein the channel attention module contains an addition layer that combines the outputs from each of the two parallel pathways.

6. The CIS of paragraph 5, wherein the channel attention module further contains a first activation function.

7. The CIS of paragraph 6, wherein the first activation function is selected from a sigmoid activation function, a rectified linear unit activation function (ReLu) layer, and/or a parametric rectified linear unit activation function (PReLu) layer, preferably a sigmoid activation function.

8. The CIS of any one of paragraphs 1 to 7, wherein the spatial attention module contains a first pooling layer and a second pooling layer, preferably wherein the first pooling layer and the second pooling layer are an average pooling layer and a max pooling layer, respectively.

9. The CIS of paragraph 8, wherein the spatial attention module contains a concatenation layer and a 3D convolutional layer after the average pooling layer and the max pooling layer.

10. The CIS of paragraph 9, wherein the spatial attention module further contains a second activation function, after the 3D convolutional layer.

11. The CIS of paragraph 10, wherein the second activation function is selected from a sigmoid activation function, a rectified linear unit activation function (ReLu) layer, and/or a parametric rectified linear unit activation function (PReLu) layer, preferably a sigmoid activation function.

12. The CIS of any one of paragraphs 1 to 11, wherein the CBAM is preceded by a convolutional layer, preferably containing a third activation function.

13. The CIS of paragraph 12, wherein the third activation function is selected from a rectified linear unit activation function (ReLu) layer, a parametric rectified linear unit activation function (PReLu) layer, and/or a sigmoid activation function layer, preferably a rectified linear unit activation function (ReLu).

14. The CIS of any one of paragraphs 1 to 13, wherein the CBAM is proceeded by a normalization layer.

15. The CIS of paragraph 14, wherein the normalization layer is selected from a batch normalization layer, a weight normalization layer, a layer normalization layer, an instance normalization layer, a group normalization layer, a batch renormalization layer, and/or a batch-instance normalization layer, preferably a batch normalization layer.

16. The CIS of any one of paragraphs 1 to 15, wherein at least one of the one or more CBAM blocks contains two or more CBAMs, each CBAM containing the channel attention module and the spatial attention module.

17. The CIS of any one of paragraphs 1 to 16, containing two or more CBAM blocks, preferably arranged in a series configuration.

18. The CIS of any one of paragraphs 1 to 17, containing a CBAM block and two fully connected layers, arranged together in series configuration,

wherein the CBAM block contains two CBAMs, each containing a channel attention module and a spatial attention module, arranged in series configuration, and

wherein each CBAM is preceded by a convolutional layer containing a ReLU activation function, and followed by a batch normalization layer.

19. The CIS of any one of paragraphs 1 to 18, wherein adjacent CBAM blocks are operably linked by a transitional layer.

20. The CIS of paragraph 19, wherein the transitional layer comprises a pooling layer.

21. The CIS of paragraph 20, wherein the pooling layer contains a max pooling layer or an average pooling layer, preferably a max pooling layer.

22. The CIS of paragraph 20 or 21, wherein the pooling layer has a stride size of (2, 2, 1) or (3, 3, 3).

23. The CIS of any one of paragraphs 1 to 22, further containing a classification layer operably linked to a terminal CBAM block of the at least one or more CBAM blocks.

24. The CIS of paragraph 23, wherein the classification layer containing a flattening layer and the two fully connected layers, preferably wherein the two fully connected layers are in a series configuration, preferably wherein the two fully connected layers comprise an activation function such as a sigmoid activation function.

25. The CIS of any one of paragraphs 1 to 24, wherein convolutional layers in subsequent CBAM blocks contain progressively more kernels than convolutional layers in prior CBAM blocks.

26. A computer-implemented method (CIM) for processing, analyzing, and/or recognizing data, the CIM involving visualizing on a graphical user interface, output from the CIS of any one of paragraphs 1 to 25.

27. The CIM of paragraph 26, wherein outputs from the parallel pathways within the channel attention module are combined and transmitted through the channel attention module's activation function, preferably a sigmoid activation function.

28. The CIM of paragraph 26 or 27, wherein input to the channel attention module is combined with output from the channel attention module's activation function, and transmitted as input to the spatial attention module.

29. The CIM of any one of paragraphs 26 to 28, wherein input to the spatial attention module is combined with output from the spatial attention module's activation function, and transmitted as input to a subsequent layer in the CBAM block.

30. The CIM of any one of paragraphs 26 to 29, wherein the data are non-contrast computed tomography (CT) medical images.

31. The CIM of any one of paragraphs 26 to 30, wherein the data are CT medical images of intra-abdominal organs (such as intra-abdominal tumoral organs) or intra-abdominal tissues (such as intra-abdominal tumoral tissues).

32. The CIM Of any one of paragraphs 26 to 31, wherein the data are CT liver scans.

33. The CIM of any one of paragraphs 26 to 32, wherein visualizing the output on the graphical user interface, provides a diagnosis, prognosis, or both, of a disease or disorder in a subject.

34. The CIM of any one of paragraphs 26 to 33, wherein the disease or disorder is hepatocellular carcinoma.

### EXAMPLES

### Example 1: Diagnosis of Hepatocellular Carcinoma with Deep Neural Networks

Hepatocellular carcinoma is a highly common and deadly cancer around the world, which is typically diagnosed using contrast-enhanced CT scans. However, the diagnostic value of non-contrast CT scans is often underestimated. This example demonstrates the feasibility of using the CBAM model to determine the presence of HCC using solely non-contrast CT scans.

Two thousand two hundred and eighty-one (2281) thin-cut computed tomography (CT) scans were retrieved from six medical centers in Hong Kong and mainland China, among which 2262 scans with non-contrast phase were included. A total of 677 patients were diagnosed as HCC and 1585 were classified as non-HCC. HCC diagnosis was based on recommendations by the American Association for the Study of Liver Disease (AASLD). The CBAM model was trained and validated on the collected CT scans, with a ratio of 7:3. In the validation cohort, the model achieved an area under curve (AUC) of 0.844 (95% CI 0.813-0.874), positive predictive value (PPV) of 0.782 (95% CI 0.705-0.859) and negative predictive value (NPV) of 0.779 (95% CI 0.745-0.813), which was comparable to the diagnostic performance using LI-RADS by experienced radiologist (AUC 0.852 [95% CI 0.820-0.881], PPV 0.975 [95% CI 0.974-0.994], NPV 0.883 [95% CI 0.855-0.909]).

In summary, the diagnostic performance of the CBAM model on non-contrast CT scans is comparable to the LI-RADS classification by radiologists. The potential of the algorithm in clinical application includes widely universal screening, diagnosing, and monitoring of high-risk populations.

Materials and Methods

(i) Description of Dataset

Two thousand two hundred and eighty-one (2281) thin-cut (<1.25 mm) triphasic CT scans were retrieved from six medical centers: Pamela Youde Nethersole Eastern Hospital (PYN), Kwong Wah Hospital (KWH), Queen Mary Hospital (QMH), Queen Elizabeth Hospital (QEH), the University of Hong Kong (HKU), and HKU-Shenzhen Hospital (SZH). A total of 2262 non-contrast scans are finally included. Diagnosis of HCC was conducted following guidelines by the AASLD, and further confirmed via clinical composite reference standard based on subsequent 12-month follow-up. All CT observations were annotated according to the LI-RADS system by a radiologist blinded to the corresponding clinical information. The data were split randomly by a 7:3 ratio into training and internal validation cohorts. In total, 677 HCC cases and 1585 non-HCC cases were divided. The training set included 465 HCC cases and 1117 non-HCC cases; the testing set included 212 HCC cases and 468 non-HCC cases. The number of cases in the various datasets is shown in Table 1.

TABLE 1
Number of HCC and Non-HCC cases in the training and testing sets
in the six medical centers (PYN, KWH, QMH, QEH, HKU, SZH).

| | Training | | Testing | |
|---|---|---|---|---|
| | HCC | Non-HCC | HCC | Non-HCC |
| PYN | 75 | 474 | 30 | 200 |
| KWH | 1 | 23 | 0 | 6 |
| QMH | 26 | 26 | 11 | 15 |
| QEH | 4 | 21 | 2 | 12 |
| HKU | 134 | 170 | 68 | 61 |
| SZH | 225 | 403 | 101 | 174 |
| Overall | 465 | 1117 | 212 | 468 |

Table 2 summarizes the numbers of liver lesions of these data sets in the training and testing sets.

TABLE 2
Number of HCC and Non-HCC liver lesions in the training and testing
sets in the six medical centers (PYN, KWH, QMH, QEH, HKU, SZH).

| | Training | | Testing | |
|---|---|---|---|---|
| | HCC | Non-HCC | HCC | Non-HCC |
| PYN | 91 | 910 | 40 | 372 |
| KWH | 1 | 47 | 0 | 12 |
| QMH | 32 | 37 | 14 | 23 |
| QEH | 6 | 32 | 4 | 21 |
| HKU | 170 | 281 | 85 | 99 |
| SZH | 242 | 689 | 108 | 304 |
| Overall | 542 | 1996 | 251 | 831 |

Non-limiting details of the architectures of the CBAM, CBAM block, and CBAM model are shown in Table 3a, Table 3b, and Table 3c, respectively.

TABLE 3a
Architecture of the convolutional block attention module (CBAM)

| | | | |
|---|---|---|---|
| Layer Type | Layer Details | Input | Output |

Channel Attention

| | | | |
|---|---|---|---|
| input | — | | |
| global_avg_pool_3d | global average pooling | input | avg_pool |
| global_max_pool_3d | global max pooling | input | max_pool |
| shared_dense1 | dense, units = channel/4 | avg_pool, max_pool | avg_pool1, max_pool1 |
| shared_dense2 | dense, units = channel | avg_pool1, max_pool1 | avg_pool2, max_pool2 |
| add | | avg_pool2, max_pool2 | add_output |
| activation | sigmoid | add_output | channel_attention_output |

Spatial Attention

| | | | |
|---|---|---|---|
| channel_avg_pool | average pooling, | channel_attention_output | channel_avg |
| | axis = channel_axis | | |
| channel_max_pool | max pooling, | channel_attention_output | channel_max |
| | axis = channel_axis | | |
| concatenate | | channel_avg, channel_max | concat_output |
| conv3d | filters = 1, kernel = 7, | concat_output | conv_output |
| | strides = 1 | | |
| activation | sigmoid | conv_output | spatial_attention_output |

TABLE 3b
Architecture of the CBAM block. cbam_module
denotes the CBAM described in Table 3a.

| Layer/Output | | |
|---|---|---|
| Name | Layer Details | Input |
| input | | |
| conv3d_1 | kernel = 3, | input |
| | activation = ‘relu’ | |
| cbam_module_1 | | conv3d_1 |
| add_1 | | conv3d_1, cbam_module_1 |
| batchnorm_1 | | add_1 |
| conv3d_2 | kernel = 3, | batchnorm_1 |
| | activation = ‘relu’ | |
| cbam_ module _2 | | conv3d_2 |
| add_2 | | conv3d_2, cbam_module_2 |
| batchnorm_2 | | add_2 |

TABLE 3c
Architecture of the complete CBAM model. cbam_block
denotes the CBAM block described in Table 3b.

| Layer/Output Name | Layer Details |
|---|---|
| input | |
| cbam_block1 | filters = 8 × factor |
| max_pool1 | pool_size = (2, 2, 1) |
| cbam_block2 | filters = 16 × factor |
| max_pool2 | pool_size = (3, 3, 3) |
| cbam_block3 | filters = 32 × factor |
| max_pool3 | pool_size = (3, 3, 3) |
| cbam_block4 | filters = 64 × factor |
| max_pool4 | pool_size = (3, 3, 3) |
| flatten | |
| dense1 | units = 512 |
| dense2 | units = 1 |
| activation | sigmoid |

(ii) Data Preprocessing

All CT scans have resolutions of 512×512 with varying number of slices. All scans were resized to 128×128 and resampled to 128 slices in order to reduce consumption of memory and computational resources. In order to better visualize and focus on the liver tissues, the intensities of all scans were truncated to within a window of [−150, 250], and then further normalized to an interval of 0 to 1.

(iii) Classification Model and Training

The Convolutional Block Attention Module (CBAM) was used to classify the liver CT scans into HCC and non-HCC. An illustration of the model and its components is shown in FIGS. 1A, 1B, and 1C. Details of the architecture are shown in Tables 3A-3C and further described below.

The CBAM model is a 3-dimensional model containing 148 layers in total and contains 65 million parameters. The CBAM model (FIG. 1A) contains four CBAM blocks connected via transitional max pooling layers. Each CBAM block (FIG. 1B) contains a convolutional layer followed by an attention module, followed by a second convolutional layer and a second attention module. Each CBAM (FIG. 1C) contains an attention module that contains a channel attention module and a special attention module.

As an object of the model is to perform a binary classification task to differentiate HCC versus non-HCC, the binary cross-entropy loss function was used for training the model. Real-time data augmentation was applied to the training dataset randomly during training, which included random flipping in the left-right and up-down direction, random 90-degree rotations, and combinations of flipping and rotation. The model was trained for 100 epochs using stochastic gradient descent (SGD) optimizer with a learning rate of 0.001. The sequence of the training dataset was randomly shuffled before each epoch.

Results

The trained CBAM model was evaluated on the internal validation dataset of 680 patients. On the validation set, the CBAM model achieved an area under curve (AUC) of 0.844 (95% CI 0.813-0.874), positive predictive value (PPV) of 0.782 (95% CI 0.705-0.859) and negative predictive value (NPV) of 0.779 (95% CI 0.745-0.813). The overall diagnostic performance of the CBAM model was comparable to that of radiologists using LI-RADS on multi-phase CT images (AUC 0.852 [95% CI 0.820-0.881], PPV 0.975 [95% CI 0.974-0.994], NPV 0.883 [95% CI 0.855-0.909]).

Those skilled in the art will recognize, or be able to ascertain using no more than routine experimentation, many equivalents to the specific embodiments of the invention described herein. Such equivalents are intended to be encompassed by the following claims.

We claim:

1. A three-dimensional computer-implemented classification system (CIS) comprising one or more Convoluted Block Attention Module (CBAM) blocks, wherein at least one of the one or more CBAM blocks comprises a CBAM, wherein the CBAM comprises a channel attention module and a spatial attention module.

2. The CIS of claim 1, wherein the channel attention module is proceeded by the spatial attention module within the CBAM.

3. The CIS of claim 1, wherein the channel attention module comprises two parallel pathways which first pass through a global average pooling layer and a global max pooling layer respectively.

4. The CIS of claim 3, wherein, within the channel attention module, each of the two parallel pathways comprises two fully connected layers.

5. The CIS of claim 4, wherein the channel attention module comprises an addition layer that combines the outputs from each of the two parallel pathways.

6. The CIS of claim 5, wherein the channel attention module further comprises a first activation function.

7. The CIS of claim 6, wherein the first activation function is selected from a sigmoid activation function, a rectified linear unit activation function (ReLu) layer, and/or a parametric rectified linear unit activation function (PReLu) layer.

8. The CIS of claim 1, wherein the spatial attention module comprises a first pooling layer and a second pooling layer, preferably wherein the first pooling layer and the second pooling layer are an average pooling layer and a max pooling layer, respectively.

9. The CIS of claim 8, wherein the spatial attention module comprises a concatenation layer and a 3D convolutional layer after the average pooling layer and the max pooling layer.

10. The CIS of claim 9, wherein the spatial attention module further comprises a second activation function, after the 3D convolutional layer.

11. The CIS of claim 10, wherein the second activation function is selected from a sigmoid activation function, a rectified linear unit activation function (ReLu) layer, and/or a parametric rectified linear unit activation function (PReLu) layer.

12. The CIS of claim 1, wherein the CBAM is preceded by a convolutional layer.

13. The CIS of claim 12, wherein the third activation function is selected from a rectified linear unit activation function (ReLu) layer, a parametric rectified linear unit activation function (PReLu) layer, and/or a sigmoid activation function layer.

14. The CIS of claim 1, wherein the CBAM is proceeded by a normalization layer.

15. The CIS of claim 14, wherein the normalization layer is selected from a batch normalization layer, a weight normalization layer, a layer normalization layer, an instance normalization layer, a group normalization layer, a batch renormalization layer, and/or a batch-instance normalization layer, preferably a batch normalization layer.

16. The CIS of claim 1, wherein at least one of the one or more CBAM blocks comprises two or more CBAMs, each CBAM comprising the channel attention module and the spatial attention module.

17. The CIS of claim 1, comprising two or more CBAM blocks.

18. The CIS of claim 1, comprising a CBAM block and two fully connected layers, arranged together in series configuration,
wherein the CBAM block contains two CBAMs, each comprising a channel attention module and a spatial attention module, arranged in series configuration, and
wherein each CBAM is preceded by a convolutional layer containing a ReLU activation function, and followed by a batch normalization layer.

19. The CIS of claim 1, wherein adjacent CBAM blocks are operably linked by a transitional layer.

20. The CIS of claim 19, wherein the transitional layer comprises a pooling layer.

21. The CIS of claim 20, wherein the pooling layer comprises a max pooling layer or an average pooling layer, preferably a max pooling layer.

22. The CIS of claim 20, wherein the pooling layer has a stride size of (2, 2, 1) or (3, 3, 3).

23. The CIS of claim 1, further comprising a classification layer operably linked to a terminal CBAM block of the at least one or more CBAM blocks.

24. The CIS of claim 23, wherein the classification layer comprises a flattening layer and the two fully connected layers.

25. The CIS of claim 1, wherein convolutional layers in subsequent CBAM blocks contain progressively more kernels than convolutional layers in prior CBAM blocks.

26. A computer-implemented method (CIM) for processing, analyzing, and/or recognizing data, the CIM involving visualizing on a graphical user interface, output from the CIS of claim 1.

27. The CIM of claim 26, wherein outputs from the parallel pathways within the channel attention module are combined and transmitted through the channel attention module's activation function, preferably a sigmoid activation function.

28. The CIM of claim 26, wherein input to the channel attention module is combined with output from the channel attention module's activation function, and transmitted as input to the spatial attention module.

29. The CIM of claim 26, wherein input to the spatial attention module is combined with output from the spatial attention module's activation function, and transmitted as input to a subsequent layer in the CBAM block.

30. The CIM of claim 26, wherein the data are non-contrast computed tomography (CT) medical images.

31. The CIM of claim 26, wherein the data are CT medical images intra-abdominal organs (such as intra-abdominal tumoral organs) or intra-abdominal tissues (such as intra-abdominal tumoral tissues)

32. The CIM of claim 26, wherein the data are CT liver scans.

33. The CIM of claim 26, wherein visualizing the output on the graphical user interface, provides a diagnosis, prognosis, or both, of a disease or disorder in a subject.

34. The CIM of claim 26, wherein the disease or disorder is hepatocellular carcinoma.
